(19) United States
(12) Patent Application Publication
(10) Pub. No.: US 2020/0384256 A1
(43) Pub. Date: Dec. 10, 2020

(54) FLUID DELIVERY ADAPTER FOR VASCULAR COMPRESSION DEVICES AND ASSOCIATED METHODS

(71) Applicant: Merit Medical Systems, Inc., South Jordan, UT (US)

(72) Inventor: Aaron Hopkinson, Herriman, UT (US)

(21) Appl. No.: 16/895,595

(22) Filed: Jun. 8, 2020

Related U.S. Application Data
(60) Provisional application No. 62/859,131, filed on Jun. 9, 2019.

(51) Int. Cl. A61M 39/10; A61M 39/20; A61M 5/178

(52) U.S. Cl. CPC A61M 39/10; A61M 39/20; A61M 2039/1077; A61M 2039/1033; A61M 5/178

(57) ABSTRACT

Fluid delivery adapters and devices used to provide hemostasis at a vascular access site are described. In some instances, the hemostasis devices include a fluid delivery adapter system configured to deliver fluid from a fluid delivery device to the hemostasis device. In some instances, the fluid delivery adapter system includes a male adapter, a cap, and a valved adapter.

### RELATED APPLICATIONS

This application claims priority to U.S. Provisional Application No. 62/859,131, filed on Jun. 9, 2019 and titled, “Fluid Delivery Adapter for Vascular Compression Devices and Associated Methods,” which is hereby incorporated by reference in its entirety.

### TECHNICAL FIELD

The present disclosure relates generally to the field of medical devices with fluid delivery lines, including compression devices configured to provide hemostasis at a vascular access puncture site. Some embodiments of the present disclosure relate to a hemostasis device used to provide hemostasis of the vasculature following vascular access as well as systems to inflate the hemostasis device.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1A is a perspective view of a fluid delivery adapter system in an unassembled state.

FIG. 1B is a perspective view of the fluid delivery adapter system of FIG. 1A in an uncoupled state.

FIG. 2 is a perspective view of the fluid delivery adapter system of FIG. 1A in a coupled state.

FIG. 3A is a top perspective view of a cap.

FIG. 3B is a bottom perspective view of the cap of FIG. 3A

FIG. 4A is a front perspective view of a male adapter.

FIG. 4B is rear perspective view of the male adapter of FIG. 4A.

FIG. 4C is a perspective longitudinal cross-sectional view of the male adapter of FIG. 4A.

FIG. 5A is a longitudinal cross-sectional view of the fluid delivery adapter system of FIG. 1A in an unassembled state.

FIG. 5B is a longitudinal cross-sectional view of the fluid delivery adapter system of FIG. 1A in a coupled state.

FIG. 6 is a perspective view of another embodiment of fluid delivery adapter system.

### DETAILED DESCRIPTION

Fluid delivery lines and systems may be part of various medical devices and used in various procedures. For examples, in some instances a hemostasis device may comprise an inflatable portion configured to provide pressure or compression when inflated with fluid though a fluid delivery line. Fluid delivery systems for other devices and applications are also within the scope of this disclosure.

Numerous medical procedures involve insertion of one or more elongate medical devices into the vasculature of a patient. Some of these interventional procedures involve delivery of a medical device through, for example, a radial artery of the patient. Achieving patent hemostasis during and/or after an interventional procedure that involves puncturing the radial artery (or other portions of the vasculature) may present certain challenges.

To facilitate hemostasis at a vascular access site, pressure may be applied directly to, adjacent to, or slightly upstream of the skin puncture site. Such pressure may prevent or reduce the leakage of blood from the vasculature access site and promote hemostasis. Certain embodiments described herein facilitate the application of pressure to promote hemostasis at a radial access site. However, the present disclosure is not so limited; the application of pressure to promote hemostasis may be applied at arteries and veins, in the wrist, hand, arm, foot, and leg, and at other vasculature access points in a patient's body.

The application of pressure at a radial access or other site may be achieved through expansion of an inflatable member, such as a bladder associated with certain embodiments of hemostasis devices. The bladder may be filled with any suitable fluid, such as air or saline, using a fluid delivery device, such as a syringe. An adapter system disposed between the bladder and the syringe may prevent accidental injection of air into an unintended medical device (e.g., intravenous infusion line) which may cause serious harm to a patient. Such an adapter system may include a delivery adapter, an adapter cap, and a valved adapter.

Embodiments may be understood by reference to the drawings, wherein like parts are designated by like numerals throughout. It will be readily understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

It will be appreciated that various features are sometimes grouped together in a single embodiment, figure, or description thereof for the purpose of streamlining the disclosure. Many of these features may be used alone and/or in combination with one another.

The phrases “coupled to” and “in communication with” refer to any form of interaction between two or more entities, including mechanical, electrical, magnetic, electromagnetic, fluid, and thermal interaction. Two components may be coupled to or in communication with each other even though they are not in direct contact with each other. For example, two components may be coupled to or in communication with each other through an intermediate component.

The directional terms “distal” and “proximal” are given their ordinary meaning in the art. That is, the distal end of a medical device means the end of the device furthest from the practitioner during use. The proximal end refers to the opposite end, or the end nearest the practitioner during use.

“Fluid” is used in its broadest sense, to refer to any fluid, including both liquids and gases as well as solutions, compounds, suspensions, blood, etc., which generally behave as fluids.

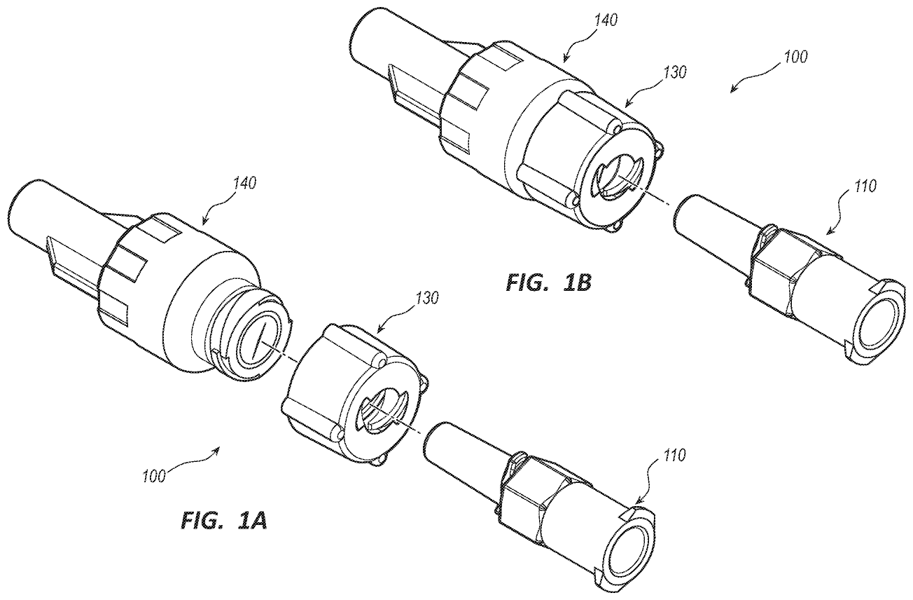

FIGS. 1-6 illustrate different views of fluid delivery adapter systems and related components. In certain views each device may be coupled to, or shown with, additional components not included in every view. Further, in some views only selected components are illustrated, to provide detail into the relationship of the components. Some components may be shown in multiple views, but not discussed in connection with every view. Disclosure provided in connection with any figure is relevant and applicable to disclosure provided in connection with any other figure or embodiment.

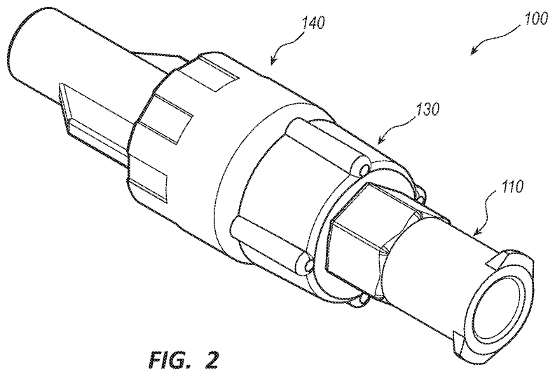

FIGS. 1A-2 depict an embodiment of a fluid delivery adapter system 100. In the illustrated embodiment as shown in FIG. 1A, the fluid delivery adapter system 100 includes a delivery adapter such as male adapter 110, an adapter cap such as cap 130, and a valved adapter 140. In FIG. 1A, these components are shown in an unassembled state. As shown in FIG. 1B, the cap 130 can be coupled to the valved adapter 140 such that the components are in an assembled state, though the male adapter 110 is not coupled to the other components in this state. FIG. 2 illustrates a coupled state, or a state in which fluid introduced at the male adapter 110 is delivered to the valved adapter 140. In the coupled state shown in FIG. 2, the male adapter 110 is coupled to the cap 130 which is coupled to the valved adapter 140. In other words, when the male adapter 110 is coupled to the cap 130 and the cap 130 is coupled to the valved adapter 140, the adapter system 100 is configured for being used to deliver a fluid to a medical device. For example, the adapter system 100 as illustrated in FIG. 2 may be used to fill a bladder of a radial artery compression device with a fluid, such that pressure or a compression force is applied at a radial artery access site.

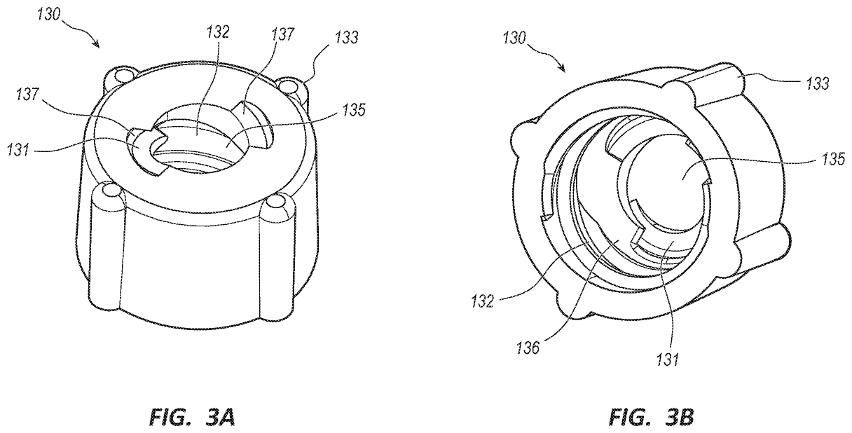

The cap 130 is shown in FIGS. 3A-3B. In the illustrated embodiment, the cap 130 is generally cylindrical in shape having a bore 136. The cap 130 includes distal internal threads 132 and proximal internal threads 131. The distal internal threads 132 are disposed adjacent a distal opening of the bore 136. The distal internal threads 132 may be configured to threadingly engage with, for example, external female luer threads of a medical female luer fitting. Embodiments where coupling features other than threads, and use of threads configured to couple to other types of fittings are likewise within the scope of this disclosure.

The proximal internal threads 131 of the cap 130 are disposed adjacent a proximal opening 135 of the bore 136 in the illustrated embodiment. An internal diameter of the proximal internal threads 131 may be smaller than an internal diameter of the distal internal threads 132. The proximal internal threads 131 may be configured to couple with the male adapter 110, as further described below. The proximal opening 135 may include slots 137 or windows providing access to the proximal internal threads 131 from a proximal surface of the cap 130. In some embodiments, the diameter of the proximal opening 135 may be sized to prevent passage of a male luer fitting through the proximal opening 135. For example, the diameter of the proximal opening 135 may be smaller than a distal diameter of a male luer protrusion such that the male luer protrusion cannot enter the cap 130 through the proximal opening 135. Embodiments where features other than threads are used for coupling the cap 130 to a delivery adapter (such as the male adapter 110) are also within the scope of this disclosure.

As noted above, the cap 130 is configured such that the proximal opening 135 is configured to prevent passage of a male luer fitting through the proximal opening 135. Thus, the cap 130 may be configured to prevent coupling of at least one end of the fluid delivery adapter system 100 with a male luer fitting. Embodiments wherein a portion the fluid delivery adapter system 100 is configured to prevent coupling with other types of connectors are likewise within the scope of this disclosure.

Gripping members 133 may be disposed on an exterior surface of the cap 130 to facilitate gripping of the cap 130 by fingers of a practitioner when the cap 130 is being coupled to the valved adapter 140. As illustrated in the depicted embodiment, the gripping members 133 may include a plurality of longitudinally oriented ridges. In other embodiments, the gripping members 133 may include any suitable grippable feature, such as ribs, grooves, bumps, dimples, recesses, a textured surface, a knurled surface, etc. The cap 130 may be formed from any suitable, rigid or semi-rigid, medical grade material, such as polycarbonate, polyethylene, polypropylene, acrylic, etc., using any suitable manufacturing technique, such as ejection molding, machining, casting, etc.

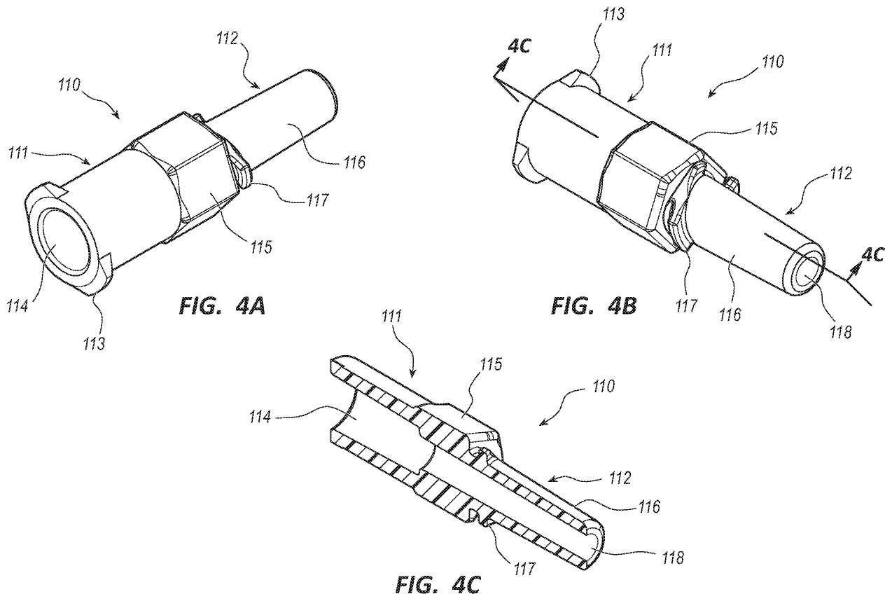

The male adapter 110, as shown in the illustrated embodiment of FIGS. 4A-4C, include a proximal portion 111 and a distal portion 112. In the illustrated embodiment, the proximal portion 111 is configured to couple to a male luer fitting. Thus, the proximal portion 111 may include a tapered bore 114 configured to sealingly receive the male protrusion of a male luer fitting. External threads or lugs 113 may be disposed on an outer surface of the proximal portion 111 adjacent a proximal end. The external threads or lugs 113 may be configured to threadingly engage with internal threads of a collar of a male luer fitting. Embodiments where the proximal portion 111 is configured with coupling features other than threads or is configured for coupling to different types of threads are likewise within the scope of this disclosure.

The distal portion 112 may include a tapered male protrusion 116 and threading lugs 117. A bore 118 may extend through the male protrusion 116 and be in fluid communication with the tapered bore 114, forming a fluid passageway through the male adapter 110. A diameter of the male protrusion 116 may be smaller than the diameter of the proximal opening 135 of the cap 130 such that the male protrusion 116 can pass through the proximal opening 135 when the cap 130 and male adapter 140 are coupled together. The threading lugs 117 may be disposed on an outer surface of the male protrusion 116 and adjacent a proximal end of the male protrusion 116. The threading lugs 117 may comprise, for example, two lugs disposed on opposing sides of the male protrusion 116. The threading lugs 117 may include a truncated helical shape, forming a partial circumference or arcs of a threaded interface around the male protrusion 116. The threading lugs 117 may be configured to pass through the slots 137 of the cap 130 and threadingly engage with the proximal threads 131 of the cap 130 when the male adapter 110 is coupled with the cap 130. Embodiments wherein the delivery adapter (such as male adapter 110) and the cap 130 are configured with different coupling features, including features other than threads and/or different types of threads are likewise within the scope of this disclosure.

A grip portion 115 may be disposed between the proximal portion 111 and the distal portion 112. As shown in the depicted embodiment, the grip portion 115 may be formed in a hexagonal shape. In other embodiments, the grip portion 115 may be formed in a triangular shape, a square shape, a pentagonal shape, or any other suitable geometric shape. In still other embodiments, the grip portion 115 may include other grip enhancing features, such as ribs, grooves, recesses, bumps, dimples, wings, textured surfaces, knurls, etc. The male adapter 110 may be formed from any suitable, rigid or semi-rigid, medical grade material, such as polycarbonate, polyethylene, polypropylene, acrylic, etc., using any suitable manufacturing technique, such as ejection molding, machining, casting, etc.

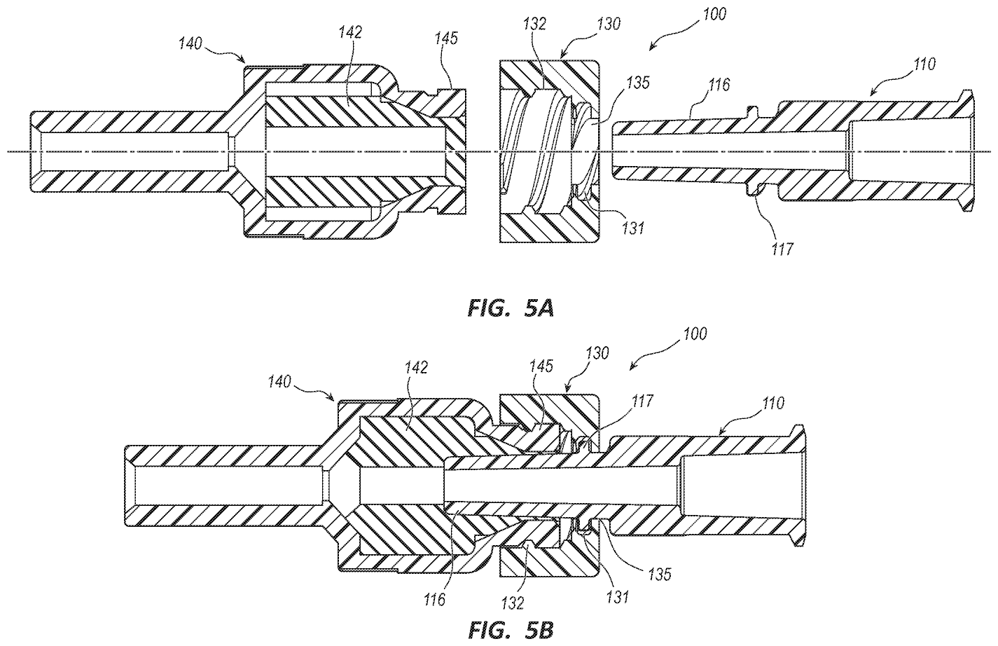

FIGS. 5A-5B depict a cross-sectional view of the fluid delivery adapter system 100. FIG. 5A depicts the fluid delivery adapter system 100 in a pre-ready or unassembled state, such as prior to coupling of the male adapter 110, the cap 130, and the valved adapter 140. FIG. 5B depicts the fluid delivery adapter system 100 in a ready or coupled state, such as following coupling of the male adapter 110, the cap 130, and the valved adapter 140 together to form a fluid pathway through the fluid delivery adapter system 100. As depicted in FIG. 5A, the male adapter 110 is shown to partially include the male protrusion 116 and the threading lugs 117. The cap 130 is shown to partially include the proximal opening 135, the proximal threads 131, and the distal threads 132. The valved adapter 140 is shown to partially include a valve member 142 configured to be actuated by the male protrusion 116 of the male adapter 110 and external threads 145 configured to threadingly engage with the distal threads 132 of the cap 130.

When the male adapter 110, the cap 130, and the valved adapter 140 are coupled together, as shown in FIG. 5B, the male protrusion 116 extends through the proximal opening 135 and into the valved adapter 140. The male protrusion 116 may actuate the valve member 142 such that fluid may be able to flow through the male adapter 110 and the valved adapter 140. The threading lugs 117 are threadingly engaged with the proximal threads 131 such that the male adapter 110 may be inhibited from inadvertent longitudinal displacement from the cap 130 and the valved adapter 140, such as due to a back pressure within the adapter system 100. The distal threads 132 of the cap 130 are threadingly engaged with the external threads 145 of the valved adapter 140. In some embodiments, the cap 130 is releasably coupled to the valved adapter 140. In other embodiments, the cap 130 is non-releasably coupled to the valved adapter 140 using any suitable technique, such as bonding, gluing, welding, etc. When the cap 130 is releasably coupled to the valved adapter 140, the cap 130 may be selectively removed from the valved adapter 140 in order to allow actuation of the valve member 142 with a male luer fitting. Embodiments wherein the features associated with the cap 130 are integrally formed as a part of the valved adapter 140 are also within the scope of this disclosure.

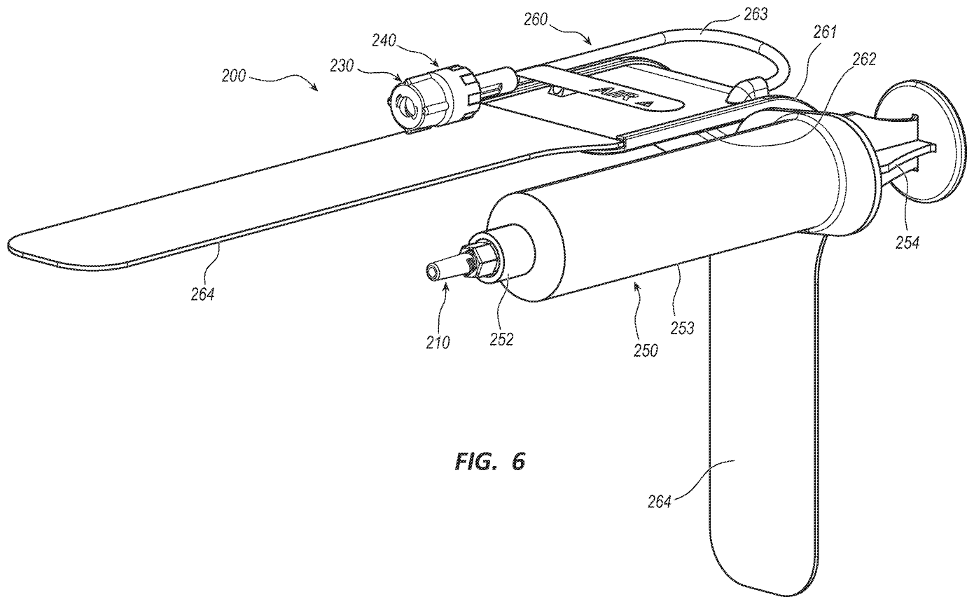

FIG. 6 illustrates another embodiment of an adapter system 200. The adapter system 200 includes a kit of components that may be used to provide hemostasis at a vascular access site, such as at a radial artery access site. As shown in the illustrated embodiment, the adapter system 200 includes a fluid delivery device 250 (such as, for example, a syringe) and a compression device 260 (in the illustrated embodiment, a compression band with an inflatable bladder). The fluid delivery device 250 may include a barrel 253 configured to retain a fluid, a plunger 254 configured to displace the fluid from the barrel 253 or to draw fluid into the barrel 253, a male luer fitting 252 disposed at a distal end of the barrel 253, and a delivery adapter, such as male adapter 210 coupled to the male luer fitting 252. In some embodiments, the male adapter 210 is releasably coupled to the male luer fitting 252. In other embodiments, the male adapter 210 is non-releasably coupled to the male luer fitting 252 using any suitable technique, such as bonding, gluing, welding, etc. Still further, embodiments where the delivery adapter (male adapter 210 in the illustrated embodiment) is integrally formed with the fluid delivery device 250 are likewise within the scope of this disclosure. The fluid delivery device 250 may be any suitable pressurized fluid delivery device, such as a pressurized IV tubing set.

In the illustrated embodiment, the compression device 260 includes a frame 261, a bladder 262, a wrist band 264, an extension tube 263, a valved adapter 240, and an adapter cap such as cap 230. The wrist band 264 may be coupled to the frame 261. The wrist band 264 may include a hook-and-loop material and be configured to secure the compression device 260 over the radial artery access site. The bladder 262 may be coupled to the frame 261. The bladder 262 may be configured to be filled with a fluid such that the bladder 262 is expanded and applies a compressive force to the radial artery access site. The extension tube 263 may be in fluid communication with the bladder 262. The valved adapter 240 may be coupled to a free end of the extension tube 263. The cap 230 may be coupled to the valved adapter 240. Embodiments wherein the features of the cap 230 are integrally formed with the valved adapter 240 and wherein both the valved adapter 240 and the cap 230 are integrally formed with a portion of the compression device 260 are also within the scope of this disclosure.

In use, the adapter system 200 may be used to apply pressure or a compressive force to a radial artery access site. The bladder 262 of the compression device 260 may be disposed over the radial artery access site and the wrist band 264 wrapped around a patient's wrist to secure the bladder 262 over the radial artery access site. The fluid delivery device 250 may be at least partially filled with a fluid (e.g., air, saline). The fluid delivery device 250 may be coupled to the compression device 250 where the male adapter 210 is coupled to the cap 230 and actuates a valve of the valved adapter 230. The fluid may be displaced from the fluid delivery device 250, through the male adapter 210, through the valved adapter 240, through the extension tube 263, and into the bladder 262 until the bladder 262 is adequately filled.

The adapter system 200 may thus be configured to reduce the risk of introducing air or other unwanted substances into a vascular access line. For example, in some procedures a vascular access line, or a line that is in communication with the vasculature, may comprise one or more standard luer fittings disposed outside the patient's body. Thus, providing a compression device configured with standard luer fittings for fluid delivery to the compression device (including delivery of air to the compression device) may present a risk of inadvertently coupling a syringe of air (intended for the compression device) to a vascular access line. Thus, a fluid delivery adapter system (100, 200) where fluid delivery to the compression device is done through fittings other than standard luer fittings may reduce the risk of inadvertent introduction of contaminants to the vasculature.

As noted above, the cap (130, 230 in the embodiments as shown) may be configured to prevent passage of a male luer fitting through the proximal opening (135, 235 in the embodiments as shown). Thus, coupling a cap (130, 230) to a compression device and/or providing a delivery adapter (such as the male adapter 110, 210) for coupling to the cap (130, 230) may comprise a portion of a method for reducing the risk of inadvertent delivery of contaminants to a vascular access line. That is, the cap (130, 230) and the delivery adapter (such as male adapter 110, 210) may be configured to couple only to each other and may have at least one end that is incompatible with direct coupling to standard fittings. This, in turn, reduces the risk of coupling a fluid delivery device intended for use with a compression device (such as fluid delivery device 250) to a vascular access line.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Similarly, in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A fluid delivery adapter system, comprising:
a delivery adapter, comprising:
a proximal portion configured to couple with a male luer fitting;
a distal portion comprising:
a tapered protrusion; and
a threaded lug disposed on a surface of the tapered protrusion;
an adapter cap, comprising:
a first section of internal threads; and
a second section of internal threads configured to threadingly couple with the threaded lug; and
a valved adapter, comprising:
external threads configured to threadingly couple with the first section of internal threads; and
a valve member disposed within the housing.

2. The fluid delivery adapter system of claim 1, wherein the proximal portion comprises:
a tapered bore configured to receive the male luer fitting; and
external threads configured to engage with internal threads of a threaded collar of the male luer fitting.

3. The fluid delivery adapter system of claim 1, wherein the distal portion further comprises a bore in fluid communication with the tapered bore.

4. The fluid delivery adapter system of claim 1, wherein the adapter cap is configured to be releasably coupled to the valved adapter.

5. The fluid delivery adapter system of claim 1, wherein the valve member is actuatable by the tapered protrusion.

6. The fluid delivery adapter system of claim 1, wherein the delivery adapter is configured to be coupled to a fluid delivery device.

7. The fluid delivery adapter system of claim 6, wherein the fluid delivery device comprises a syringe.

8. The fluid delivery adapter system of claim 1, wherein the adapter cap further comprises a proximal opening configured to prevent passage of a male luer fitting through the opening.

9. The fluid delivery adapter system of claim 8, wherein the tapered protrusion is configured to be disposed through the proximal opening.

10. An adapter cap, comprising:
distal internal threads;
proximal internal threads, wherein an inner diameter of the proximal internal threads is less than an inner diameter of the distal internal threads; and
a proximal opening.

11. The adapter cap of claim 10, wherein the distal internal threads are configured to threadingly couple with external threads of a female luer fitting.

12. The adapter cap of claim 10, wherein the proximal internal threads are disposed adjacent the proximal opening.

13. The adapter cap of claim 10, wherein the proximal opening is configured to prevent passage of a male luer fitting through the proximal opening.

14. A method of reducing the risk of inadvertent coupling of a fluid delivery device to a vascular access line, the method comprising:
providing a fluid delivery device with a delivery adapter; and
providing a compression device with an adapter cap configured to couple to the delivery adapter;
wherein the delivery adapter is configured to couple to the cap;
wherein at least one end of the adapter cap is not configured to couple directly to a standard luer fitting; and
wherein at least one end of the delivery adapter is not configured to couple directly to a standard luer fitting.

15. The method of claim 14, wherein the delivery adapter comprises:
a tapered protrusion; and
a threaded lug disposed on a surface of the tapered protrusion.

16. The method of claim 14, wherein the adapter cap comprises:
distal internal threads;
proximal internal threads, wherein an inner diameter of the proximal internal threads is less than an inner diameter of the distal internal threads; and
a proximal opening.
